(19) United States
(12) Patent Application Publication
(10) Pub. No.: US 2002/0098139 A1
(43) Pub. Date: Jul. 25, 2002

(54) Resposable sterilization and transport unit

(76) Inventor: Beverly Sparks, Oceanside, CA (US)

Correspondence Address: Sam Talpalatsky, Esq., 10867 Cloverhurst Way, San Diego, CA, 92130, US

(21) Appl. No.: 09/767,485

(22) Filed: Jan. 22, 2001

(51) Int. Cl. A61L 2/00; A61L 9/00

(52) U.S. Cl. 422/300; 422/292

(57) ABSTRACT

Disclosed is a resposable container usable for storage, transport, disposal and sterilization of contents. The container generally provides an area defined by a series of side panels, a floor panel and a top panel. At least one selectively openable exhaust vent, placed in the generally lower portion of one or more of the panels, allows sterilant, injected into the container from a generally higher point, to exhaust the contaminated or non-sterile gases. A laminate selected for functional compatibility with the user preferred sterilization method covers at least one of the sides of one of the panels of the container.

### FIELD OF THE INVENTION

The field of the present invention relates generally to a resposable (reusable and disposable) container used for wrapped and unwrapped product sterilization, aseptic sterile product storage, transport, shelf-life protection, decontamination and disposal of bio-hazardous materials.

### BACKGROUND ART

Often surgical instruments and materials (hereafter the use of the term “instruments” or “materials” will have interchangeable meaning) used in hospitals, medical clinics, research and medical laboratories, sterilization centers, medical, dental and veterinarian offices and the like are sterilized and used outside of the room where such instruments and materials are sterilized. The path from the sterilization location is almost always non-sterile. Once instruments and/or materials are sterilized, they must be protected from exposure to airborne and handling contamination from the sterilizer to the point of use. Items that are wrapped for post-sterilization protection from airborne and handling contamination, have a limited time and event related shelf life beyond which they are considered unsafe for use. Sterile products may be sent to many various locations and environments for possible emergency use or for routine standby, in the event of need. If not utilized, these products are usually returned to a sterile supply room for future use. Inadvertent contamination of sterile items can take place through airborne particles, dropping, spilling of procedural solutions, crushing or tearing of wrapping materials or penetration with sharp objects. Although many precautions may be implemented to minimize the exposure to contaminants, it is difficult to maintain a germ free environment. Wrapping materials alone do not provide adequate contamination protection against inadvertent contamination and contaminated instruments and/or materials must undergo a sterilization reprocessing or disposal.

After use, soiled instruments or materials are often placed back into the same containers that were used to store and transport them in sterilized form. Some of these containers carry their contents to a cleaning facility where the container, instruments and reusable materials go through decontamination, cleaning process and sterilization. Others transport bio-hazardous materials such as knife blades, suture and hypodermic needles as well as other contaminates to designated bio-hazardous disposals where they are discarded. This cycle is repeated for the life of the reusable container, instruments and other materials.

### SUMMARY OF THE INVENTION

The device of the present invention is an aseptic, resposable sterilization and transport container that overcomes the shortcomings of the prior art. In one embodiment it is enclosable and it provides a user convertible aseptic transport of sterilized materials to a procedure. The same device as a safety product, can also be utilized to isolate used sharps such as needles and knife blades and to collect contaminated materials. The contaminated biomaterials may thereafter be discarded along with the container. Yet in another mode, when not contaminated with bio-waste items, the device, with its laminated or otherwise non-porous construction may be reused as a sterilization container.

The resposable container of the present invention may be constructed of metalized fiberboard, or laminated metalized cardboard or plastics adhered together with means known in the art such as adhesive, radio frequency welds, rivets or fasteners. The non-metalized version may be constructed of non-porous chemically and heat resistant materials such as plastics as an example. In one embodiment, it is constructed in a form of a box having a lid, into which materials are placed for sterilization, storage and transport. Its laminations are consistent with the anticipated form of sterilization method desirable for its contents. The container of the present invention is initially shipped and stored flat to conserve space. When needed, the container is constructed. When its service is completed, it is discarded.

In one form, the container of the present invention may serve as a sterilization unit. When sterilized with a sterilization agent (sterilant), such as steam or a high temperature sterilization agent as an example, the exhaust vent, which is preferably placed toward the bottom of one of the panels of the container, is selectively enabled providing an exhaust path for the sterilization agent. This allows a faster sterilization of the contents. The method for flash sterilization is described further in Applicant's U.S. Pat. No. 4,663,122, which is fully incorporated herein by reference.

Further, the device of the present invention may be used with a number of sterilization methods and apparatus. For example, the device of the present invention may be applied and used with sterilization systems and processes such as microwave, steam-microwave, electron beam irradiation (e-beam), ultraviolet (UV) light, dry heat, convection heat, convection steam, gamma irradiation, hydrogen peroxide, ethylene oxide, ozone, steam (high speed gravity displacement) unwrapped method, steam gravity displacement unwrapped and wrapped methods, steam pulse-vacuum wrapped or unwrapped, steam pre-vacuum wrapped or unwrapped, paracetic acid, chlorine dioxide, gas plasma, formaldehyde-low temperature steam, microwave—bactericide, xenon lamp (broad spectrum pulsed-light), glass bead, vacuum ovens, heat conduction ovens, forced air ovens, solvent venting ovens, anprolene gas. Depending on the system, the panels of the present invention are coated, sandwiched, covered, lined, laminated or constructed with available laminates or materials suitable or compatible with the desired sterilization system and/or process.

In one embodiment, the device of the present invention is constructed in a form of a container having a top through which materials are placed for sterilization, storage, transport and/or disposal. Its construction, inner lining or laminate is consistent with the anticipated form of sterilization desirable for its contents. Depending on the sterilization process, the outer skin and the construction material of the container may be similarly coated, covered, lined, laminated or constructed. The container of the present invention is initially shipped and stored flat to conserve space. When needed, the container is constructed. When its service is completed, it is discarded.

Other aspects, advantages and novel features of the present invention will become apparent from the following Detailed Description, when considered in conjunction with the accompanying drawings.

### BRIEF DESCRIPTION OF THE DRAWINGS

FIG. 1 is an elevated view of one of the embodiments of the present invention.

FIG. 2 is a cutaway view of one of the panels of the present invention.

FIG. 3 is an elevated view of a rack for support of contents in the present invention.

FIG. 4 is an elevated view of a sterilization appliance together with the device of the present invention.

### DETAILED DESCRIPTION

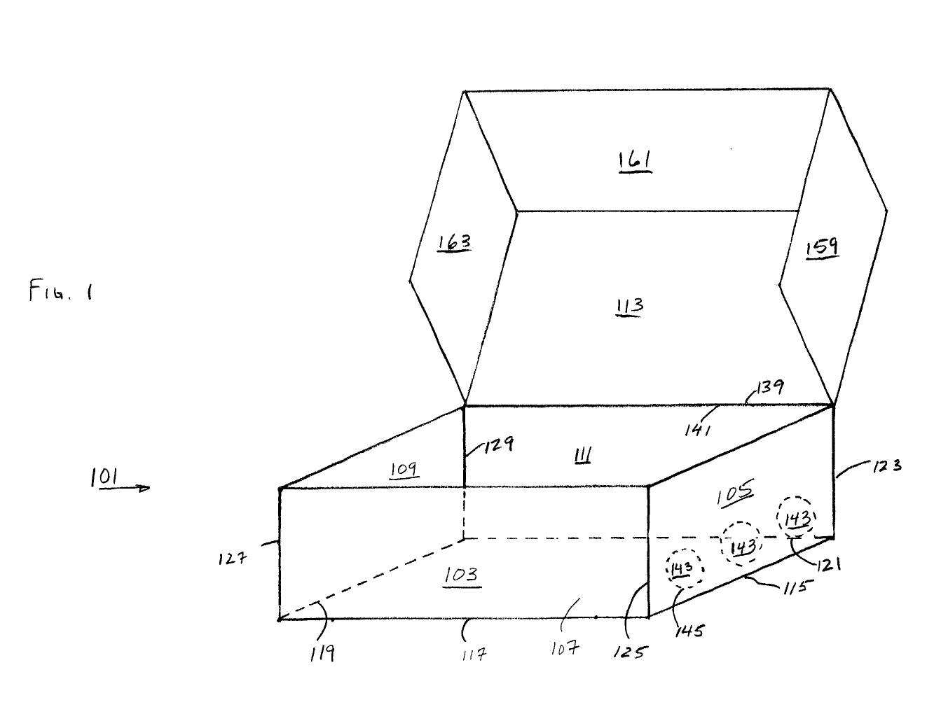

An elevated view of the device 101 of the present invention is shown in FIG. 1. In its rectangular embodiment, device 101 consists of a bottom panel 103 having marginal edges 115, 117, 119 and 121. Connected to marginal edges 115, 117, 119 and 121 of panel 103 are side panels 105, 107, 109 and 111 along their respective marginal edges. As shown in FIG. 1, panels 105, 107, 109 and 111 are further connected to each other along their respective marginal edges 123, 125, 127 and 129. In one embodiment, a top panel 113 is movably or hingedly attached to the marginal edge 141 of side panel 111 along its marginal edge 139. Top panel 113 is connected to its respective side panels 159, 161 and 163. In another embodiment of the present invention (not illustrated), top panel 113 with or without one or more of its respective side panels 159, 161 and 163 is removable from container 101 and further comprises a panel placeable on the top of and/or covering side panels 103, 105, 107, 109 and 111. Although described in its rectangular embodiment, device 101 may be of any shape including, without limitation, square, oblong, rectangular, ovoid, or round and its dimensions may be variable and several.

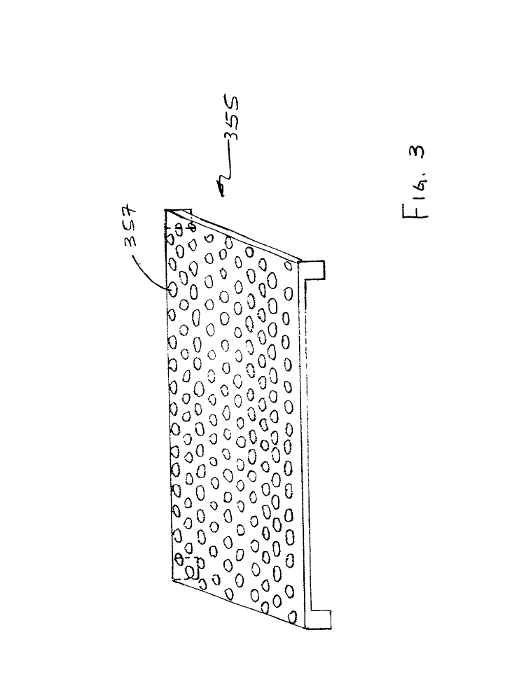

In the embodiment of the invention where device 101 is used as a sterilization container, it is useful to employ at least one exhaust vent 143 in one or more side panels 105, 107, 109 and/or 111. In this embodiment it may be desirable to remove the contaminated or non-sterile atmosphere in the container 101 by injecting a sterilizing agent/sterilant such as high temperature gas, steam, chemical or other sterilizing agents, as examples, into container 101 through its top portion. Exhaust vent 143 facilitates flow of sterilizing agent(s) through container 101, over the instruments or materials that are supported by a rack 355 (illustrated in FIG. 3). Since device 101 is multifunctional and it may not be desirable to have an exhaust vent open for applications such as transport or biohazard transport, exhaust vent 143 is defined by a series of perforations or scores 145 (which terms are used interchangeably). When it is desirable to employ exhaust vent 143, the user can readily enable exhaust vent 143 by separating the panel material along the perforations or scores 145. In the embodiment shown in FIG. 1, perforations or scores 145 allow the user to remove the material partially or fully from device 101. The scored or perforated sides designating and facilitating one or more vents 143 may vary in size, shape and configuration. They may be of any shape including, without limitation, square, rectangular, round, oblong, or ovoid and they may be variably located on one or more panels of device 101. In other embodiments perforations 145 can define a number of shapes and functions known in the art including, without limitation, a continuous or non-continuous loop allowing the user to selectively open and close vent(s) 143 without fully removing it/them. In other embodiments of the invention (not illustrated) exhaust vent(s) 143 maybe achieved by cutting out an opening or having a selective opening such as a door or a subpanel. Yet in other embodiments, exhaust vent 143 may be enabled by peeling away or separating a cover element removably placed over an opening.

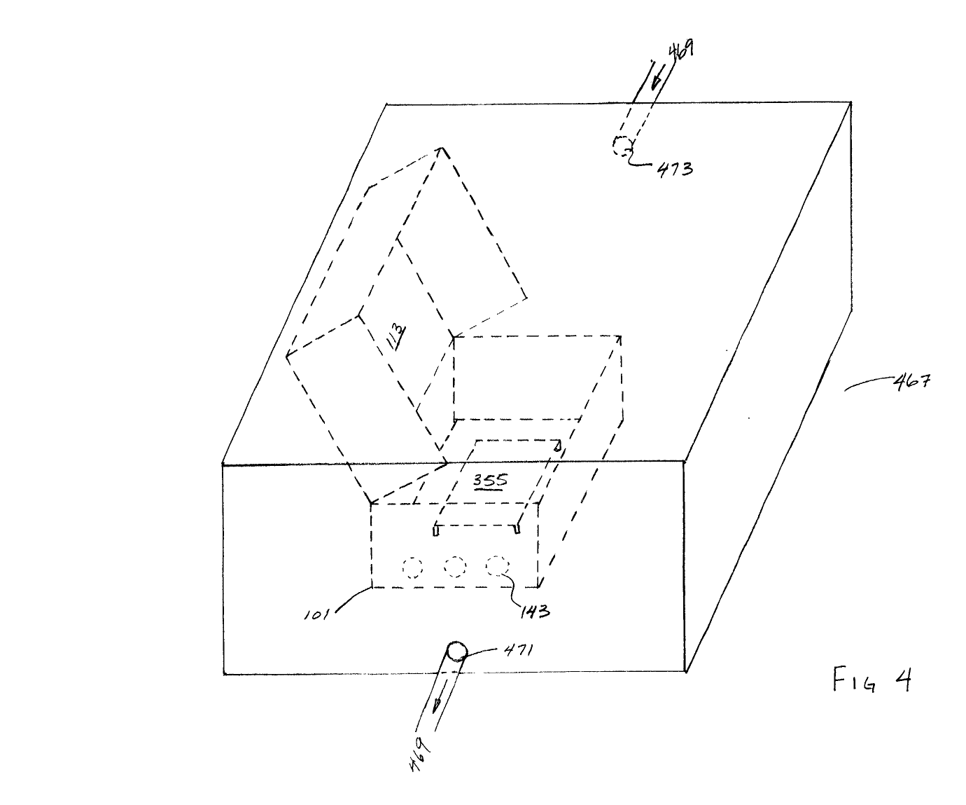

As illustrated in FIG. 4, in sterilization appliances 467 using a gaseous or heat sterilant 469, device 101 with its top panel 113 in the open position, is placed inside appliance 467. Sterilant 469 is then applied inside appliance 467 through an entry 473 descending over the instruments and/or materials placed on rack 355. For best and most expedient results, the aggregate area defined by one or more vents 143 should equal or be greater than the area of exhaust 471 of appliance or sterilizer 467. This eliminates cold spots from forming in device 101, which prevent or retard the sterilization of the materials. Generally, device 101 is placed with exhaust(s) 143 matched to or in the general vicinity of exhaust 471. Accordingly, exhaust 471 is generally positioned toward the mid portion of one of the side panels of sterilizer 467. Some sterilizers 467 assist the flow of the sterilizing agent(s) with a siphon or some type of flow assist, thereby facilitating the flow of the sterilizing agent(s) through sterilizer 467 and device 101 chambers to provide a more expedient and/or more efficient sterilization process.

It will be recognized by those skilled in the art that steam and high temperature sterilization techniques have target temperatures substantially between 250 and 276 degrees Fahrenheit. This achieves at least two things. First, the relatively high temperature and/or moisture raised to high temperature kills germs and secondly, they alone or in combination provide positive pressure within sterilizier 467 and device 101 chambers as long as the chambers' temperature is higher than ambient temperature outside the chambers. Thus, contaminants that may be introduced through handling of device 101 and its contents after the sterilization cycle are either destroyed or are pushed out of the chambers through the positive pressure.

Furthermore, where device 101 is applied with steam sterilization techniques, in one of the embodiments of the present invention, the panels of device 101 are constructed of generally disposable materials and are sandwiched with a metalized coating or layer. The metal could be aluminum, although any generally corrosion resistant metal, alloy or material capable of withstanding high temperature and moisture could be employed. Where device 101 is applied with microwave, pulsed light, or electro beam sterilization techniques, in one of the embodiments of the present invention, the panels of device 101 are generally constructed of paper, fiber, plastic, or generally disposable materials that do not have an negative reaction when exposed to the described sterilization means. Yet in a third example, where device 101 is applied with acidic or corrosive sterilizing agents including, without limitation, parasitic acids or hydrogen peroxide, the panels of device 101 are generally constructed of plastic or fiber and/or are sandwiched with a metal, alloy or otherwise a material capable of withstanding the corrosive effects of the agents. One skilled in the art will select or choose from a number of materials or laminates to cover disposable materials that could comprise or cover panels of device 101 consistent with the requirements of a sterilization process employed.

As shown in FIG. 3, rack 355 has a plurality of perforations or openings 357. The perforations or openings can be of any number, shapes and dimensions or combination thereof, as long as rack 355 continues to substantially support and elevate the instruments and materials from bottom panel. As described above, rack 355 facilitates the sterilization process. As the panels, rack 355 is constructed or covered from materials substantially the same as device 101, or compatible with device 101 as disclosed in this specification, or where the instruments are heavy, rack 355 may be constructed of a metal or alloy consistent with the sterilization process in which it is employed. In applications where the instruments or materials placed in device 101 are too heavy for its structure, device 101 may be architected and designed to support the heavier contents by means known in the art. Additionally, the user may apply a tray to support device 101 and its contents.

Device 101 of the present invention may also be utilized as an aseptic storage and/or transport unit. The sterilized and usually wrapped materials are placed in device 101 and are placed in storage. The device of the present invention protects and prolongs the shelf life by preventing airborne particles, moisture and bellows-effect contamination from contaminating the sterilized contents. As described above, where device 101 is used in sterilization and its vent(s) 143 are opened, flaps 159, 161 and 163 cover vent(s) 143 thereby limiting airborne contaminants from entering into device 101 and contaminating its sterilized contents. Accordingly, one or more flaps 159, 161 and 163 should have dimensions sufficient to cover exhaust vent(s) 143. Further, in other embodiments of the present invention, at least one or more flaps 159, 161 and 163 could be attachable to its respective panels 115, 117, 119 or 111. A mild adhesive, tape, hasp and loop, or a hook and loop combination (such as Velcro®) as examples can serve as an exterior sterilization indicator and validation, facilitate attachment and prevent accidental opening and exposure.

When the materials are called to service or are generally moved, device 101 provides an excellent transport facility, protecting its sterile contents from contaminants including, without limitation, airborne contaminants such as germs. As described in more detail above, the positive pressure within the chamber of device 101 allows a non-sterile technician to remove device 101 along with its sterilized items from sterilizer 467 and transport device 101 and its contents to the point of use. This eliminates the need for the risky practice of requiring the sterile scrub assistant to enter a non-sterile location to retrieve these items, and becoming contaminated in the process.

Additionally, the device of the present invention allows the sterilized materials to remain on standby for emergency sterilization of non-sterile instruments or materials. In this mode, once the sterilized materials are delivered, the staff may choose to flash sterilize the contents with steam or high temperature (autoclave) agents, as an example. In another instance, the staff may wish to validate or revalidate the sterilization. As an example, the user would optionally remove some of the material from one or more panels 105, 107, 109 and/or 111 using perforations 145 creating exhaust vent(s) 143 and sterilize the contents again as described above.

Sterilization monitoring methods are generally employed for cycle process validation. Each type of validation device (integrator) monitors a different set of parameters. The two basic types of tests are biological and chemical. For example, ethylene oxide and anprolene gas sterilizers, employ live spore biological testing devices utilizing Bacillus Subtilis organisms, plus gas sensitive color changing chemical sterilization integrator strips. For steam sterilization monitoring, biological testing with live spores of Bacillus Stearothermophilus, and chemical color changing sterilization integrator strips monitor the steam sterilant exposure parameters for each cycle. Vent(s) 143 of device 101 provide access to its interior/chamber for optimal placement of the spore biological tests and the chemical sterilization integrators. Through vent(s) 143 the extended portion of the monitoring devices protrudes exterior to device 101 for selective monitoring and device removal. This feature permits immediate access to sterilization process validation prior to lid closure with sealing off of vents 143, and prior to removing device 101 and sterile contents from the sterilizer unit 467. If vent(s) 143 are not employed for the monitoring process as described, aseptic removal of the monitoring devices would be delayed until their removal at the point of use. At the time of anticipated use, if the monitoring device indicates incomplete sterilization, the contents must be re-sterilized. This undesirable circumstance may occur in a location remote from the sterilizing location causing a potentially life threatening delay, which could have been avoided if the vent(s) 143 had been employed for chemical test strip retrieval from the sterilized invention for monitoring results, prior to removal from the sterilizer 467.

As mentioned above, device 101 used in a high temperature sterilization process facilitates the use of the integrators, as the high temperature provides positive pressure within the chamber of device 101 and thus rejects and/or destroys the contamination introduced by the user removing the integrator after the sterilization cycle. Yet in another embodiment, a view panel is integrated into one or more panels of device 101, allowing the user to view the validation indicators. Thereafter, sterilized materials can be used, or as previously described, they can be stored in device 101 until required for a procedure.

As discussed above, during and after the medical procedure, device 101 can be utilized as a receptacle and transport container for contaminated, biohazardous materials. The contaminated materials are placed in device 101 to limit the spread of the biohazardous materials and to provide a safe and efficient transport protecting the medical staff, bystanders and the environment from the contaminated materials. Additionally device 101 allows disposal or incineration of the biohazardous materials together with device 101, without additional cleaning, sterilization, processing or segregation.

Also as mentioned above, in one embodiment of the present invention, device 101 is resposable. It is designed for reuse or a single use and thus it is constructed from disposable materials such as cardboard, plastic, fiber, coated aluminum, with vinyl or epoxy matrix and the like. Having resposable device 101 allows the user to dispose of contaminated containers without having to sterilize them. This is advantageous because many sterilants and cleaning agents are corrosive and they degrade the integrity of the sterilization containers. This phenomenon leads to crazing and cracking of plastics, with pitting and corrosion of the containers made of materials as strong as stainless steel disturbing the smooth surface of these otherwise durable materials, and creating a safe haven for bacterial and other contaminants. Similar results are experienced with other materials such as plastics and laminates. Pitting, crazing corrosion and phenomenon disturbing the smooth, continuous plane of the container provides a safe haven for bacteria and contaminants and causes containers to lose their utilitarian function. Accordingly, disposing of device 101 is a cleaner, more economic and attractive alternative over disposing conventional containers made of stainless steel or alloys.

Device 101 can also be utilized as a receptacle for contaminated instruments. Generally, once the instruments are contaminated or soiled, they are placed in device 101 and are transported to a cleaning and sterilization facility. In transport, device 101 shields the environment from biohazardous contamination. Thereafter, device 101 can be decontaminated and sterilized, disposed or placed back in service.

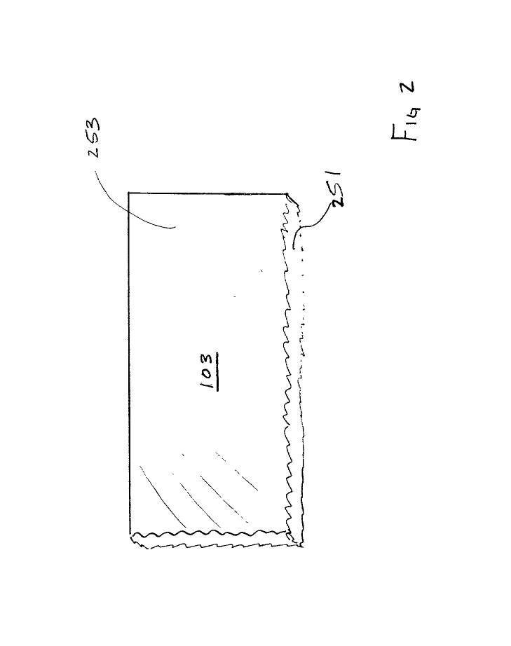

As shown in FIG. 2, at least one of the panels 103, 105, 107, 109, 111 and 113 are laminated or constructed from a disposable material 251 such as cardboard, plastic, fiberglass, styrofoam, aluminum or like disposable materials known in the art. These materials preferably have sufficient characteristics to support the structure of device 101 or are formed or constructed in ways known in the art to provide the requisite support and structure. Additionally or alternatively, one or more panels 103, 105, 107, 109, 111 and 113 may be laminated coated with a material 253. Material 253 may be chosen by its characteristics, utility, expense and/or function and for compatibility with sterilization/decontamination technologies such as microwave, steam-microwave, electron beam irradiation (e-beam), ultraviolet (UV) light, dry heat, convection heat, convection steam, gamma irradiation, hydrogen peroxide, ethylene oxide, ozone, steam (high speed gravity displacement) unwrapped method, steam gravity displacement unwrapped and wrapped methods, steam pulse-vacuum wrapped or unwrapped, steam pre-vacuum wrapped or unwrapped, paracetic acid, chlorine dioxide, gas plasma, formaldehyde-low temperature steam, microwave—bactericide, xenon lamp (broad spectrum pulsed-light), glass bead, vacuum ovens, heat conduction ovens, forced air ovens, solvent venting ovens, anprolene gas. As an example, a facility may choose laminate 253 by its compatibility with steam or e-beam sterilization or microwave sterilization. In other embodiments, one or more panels of device 101 may be fully or partially constructed from a material compatible with the above sterilization processes.

In one embodiment (not shown) device 101 is shipped and stored in a flat form. When a new device 101 is needed, it is constructed. Thus, a number of containers 101 can be easily and efficiently stored in a small space. Top flap 113 can either be attached to one of the side panels 105, 107, 109 or 111 or it can be detached. In either mode, flap 113 encloses device 101 for clean transport and storage.

While embodiments and implementations of the subject invention have been shown and described, it should be apparent that many more embodiments and implementations are within the scope of the subject invention. Accordingly, the invention is not to be restricted, except in light of the claims and their equivalents.

I claim:

1. A resposable container usable for storage, transport, disposal and sterilization of contents, the container comprising:
a) an enclosable area comprised of a series of side panels, a floor panel and a top panel;
b) at least one selectively openable exhaust vent in at least one of the panels; and
c) a laminate covering at least one side of at least one of the panels, where the laminate is compatible with a sterilization process used to sterilize the contents of the container.

2. The resposable container of claim 1 wherein the top panel is hingedly attached to one of the side panels.

3. The resposable container of claim 1 wherein the top panel is hingedly attached to one of the side panels and the top panel further comprises at least one flap extending from the top panel to cover at least one side panel and the exhaust vent therein.

4. The resposable container of claim 1 wherein the exhaust vent is defined by perforations allowing the user to selectively open the exhaust vent.

5. The resposable container of claim 1 further comprising a rack elevating the contents of the container from the floor panel.

6. The resposable container of claim 5 wherein the rack has a number of passages allowing a sterilant to access the rack and the contents thereon.

7. The resposable container of claim 1 wherein the sterilization process is chosen from a group comprising at least one of microwave, steam-microwave, electron beam irradiation, irradiation, ultraviolet light, dry heat, convection heat, convection steam, gamma irradiation, hydrogen peroxide, ethylene oxide, ozone, steam unwrapped method, steam gravity displacement wrapped method, steam pulse-vacuum wrapped or unwrapped, steam pre-vacuum wrapped or unwrapped, paracetic acid, chlorine dioxide, gas plasma, formaldehyde-low temperature steam, microwave—bactericide, xenon lamp, glass bead, vacuum ovens, heat conduction ovens, forced air ovens, solvent venting ovens, anprolene gas.

8. A resposable container useable for storage, disposal, transport and sterilization of contents, the container comprising:
a) an inner area defined by a bottom panel, a series of side panels and a top panel providing selectable entry to the inner area of the container;
b) at least one selectable exhaust vent in at least one of the panels, providing exhaust of a sterilant sterilizing the contents placed in the inner area;
c) a laminate covering at least one side of at least one of the panels, where the laminate is compatible with a sterilization process used to sterilize the contents of the container; and
d) wherein the container protects the contents from contamination during storage.

9. The resposable container of claim 8 wherein the top panel is hingedly attached to one of the side panels.

10. The resposable container of claim 8 wherein the top panel is hingedly attached to one of the side panels and the top panel further comprises at least one flap extending from the top panel to cover at least one side panel and the exhaust vent therein.

11. The resposable container of claim 8 wherein the exhaust vent is defined by perforations allowing the user to selectively open the exhaust vent.

12. The resposable container of claim 8 further comprising a rack where the rack has a number of passages allowing the sterilant to access the rack and the contents thereon.

13. The resposable container of claim 8 wherein the sterilization process is chosen from a group comprising at least one of microwave, steam-microwave, electron beam irradiation, irradiation, ultraviolet light, dry heat, convection heat, convection steam, gamma irradiation, hydrogen peroxide, ethylene oxide, ozone, steam unwrapped method, steam gravity displacement wrapped method, steam pulse-vacuum wrapped or unwrapped, steam pre-vacuum wrapped or unwrapped, paracetic acid, chlorine dioxide, gas plasma, formaldehyde-low temperature steam, microwave—bactericide, xenon lamp, glass bead, vacuum ovens, heat conduction ovens, forced air ovens, solvent venting ovens, anprolene gas.

14. A resposable container useable for storage, transport, disposal and sterilization of contents, the container comprising:
a) a series of side panels attachable along one of their marginal edges to respective marginal edges of a bottom panel and along its other marginal edges attachable to adjacent side panels, together the panels comprising an inner area of the resposable container;
b) a top panel providing selectable entry to the inner area for placement of contents therein;
e) at least one selectable exhaust vent in at least one of the panels, providing exhaust of a sterilant sterilizing the inner area; and
f) a laminate placed on at least one of the bottom panel, top panel and inner surface of the walls to protect the contents from contamination.

15. The resposable container of claim 14 wherein the top panel is hingedly attached to one of the side panels.

16. The resposable container of claim 14 wherein the top panel is hingedly attached to one of the side panels and the top panel further comprises at least one flap extending from the top panel to cover at least one side panel and the exhaust vent therein.

17. The resposable container of claim 14 wherein the exhaust vent is defined by a series of perforations allowing the user to selectively open the exhaust vent.

18. The resposable container of claim 14 further comprising a rack elevating the contents of the container from the floor panel.

19. The container of claim 18 wherein the rack has a number of passages allowing the sterilant to access the rack and the contents thereon.

20. The resposable container of claim 14 wherein the sterilization process is chosen from a group comprising at least one of microwave, steam-microwave, electron beam irradiation, irradiation, ultraviolet light, dry heat, convection heat, convection steam, gamma irradiation, hydrogen peroxide, ethylene oxide, ozone, steam unwrapped method, steam gravity displacement wrapped method, steam pulse-vacuum wrapped or unwrapped, steam pre-vacuum wrapped or unwrapped, paracetic acid, chlorine dioxide, gas plasma, formaldehyde-low temperature steam, microwave—bactericide, xenon lamp, glass bead, vacuum ovens, heat conduction ovens, forced air ovens, solvent venting ovens, anprolene gas.

21. A resposable container usable for storage, transport, disposal and sterilization of contents, the container comprising:
a) an enclosable area comprised of a series of side panels, a floor panel and a top panel;
b) at least one selectively openable exhaust vent in at least one of the panels; and
c) wherein the resposable container is comprised of a material means for compatibility with a sterilization process used to sterilize its contents.

22. The resposable container of claim 21 wherein the top panel is hingedly attached to one of the side panels.

23. The resposable container of claim 21 wherein the top panel is hingedly attached to one of the side panels and the top panel further comprises at least one flap extending from the top panel to cover at least one side panel and the exhaust vent therein.

24. The resposable container of claim 21 wherein the exhaust vent is defined by a series of perforations allowing the user to selectively open the exhaust vent.

25. The resposable container of claim 21 further comprising a rack elevating the contents of the container from the floor panel.

26. The resposable container of claim 25 wherein the rack has a number of passages allowing the sterilant to access the rack and the contents thereon.

27. The resposable container of claim 21 wherein the sterilization process is chosen from a group comprising at least one of microwave, steam-microwave, electron beam irradiation, irradiation, ultraviolet light, dry heat, convection heat, convection steam, gamma irradiation, hydrogen peroxide, ethylene oxide, ozone, steam unwrapped method, steam gravity displacement wrapped method, steam pulse-vacuum wrapped or unwrapped, steam pre-vacuum wrapped or unwrapped, paracetic acid, chlorine dioxide, gas plasma, formaldehyde-low temperature steam, microwave—bactericide, xenon lamp, glass bead, vacuum ovens, heat conduction ovens, forced air ovens, solvent venting ovens, anprolene gas.

28. A resposable container usable for storage, transport, disposal and sterilization of contents, the container comprising:
a) an enclosable area comprised of a series of side panels, a floor panel and a top panel;
b) at least one selectively openable exhaust vent in at least one of the panels; and
c) wherein the container is comprised of a material compatible with a sterilization process used to sterilize its contents.

29. The resposable container of claim 28 wherein the top panel is hingedly attached to one of the side panels.

30. The resposable container of claim 28 wherein the top panel is hingedly attached to one of the side panels and the top panel further comprises at least one flap extending from the top panel to cover at least one side panel and the exhaust vent therein.

31. The resposable container of claim 28 wherein the exhaust vent is defined by a number of perforations or scores allowing the user to selectively open the exhaust vent.

32. The resposable container of claim 28 further comprising a rack elevating the contents of the container from the floor panel.

33. The resposable container of claim 32 wherein the rack has a number of passages allowing the sterilant to access the rack and the contents thereon.

34. The resposable container of claim 28 wherein the sterilization process is chosen from a group comprising at least one of microwave, steam-microwave, electron beam irradiation, irradiation, ultraviolet light, dry heat, convection heat, convection steam, gamma irradiation, hydrogen peroxide, ethylene oxide, ozone, steam unwrapped method, steam gravity displacement wrapped method, steam pulse-vacuum wrapped or unwrapped, steam pre-vacuum wrapped or unwrapped, paracetic acid, chlorine dioxide, gas plasma, formaldehyde-low temperature steam, microwave—bactericide, xenon lamp, glass bead, vacuum ovens, heat conduction ovens, forced air ovens, solvent venting ovens, anprolene gas.

35. A resposable container usable for storage, transport, disposal and sterilization of contents, the container comprising:
a) an enclosable area comprised of a series of side panels, a floor panel and a top panel;
b) at least one selectively openable exhaust vent in at least one of the panels; and
d) a laminate covering at least one side of at least one of the panels, where the laminate is compatible with a sterilization process used to sterilize the contents of the container and where the sterilization process is chosen from a group comprising at least one of microwave, steam-microwave, electron beam irradiation, irradiation, ultraviolet light, dry heat, convection heat, convection steam, gamma irradiation, hydrogen peroxide, ethylene oxide, ozone, steam unwrapped method, steam gravity displacement wrapped method, steam pulse-vacuum wrapped or unwrapped, steam pre-vacuum wrapped or unwrapped, paracetic acid, chlorine dioxide, gas plasma, formaldehyde-low temperature steam, microwave—bactericide, xenon lamp, glass bead, vacuum ovens, heat conduction ovens, forced air ovens, solvent venting ovens, anprolene gas.

36. The resposable container of claim 35 wherein the top panel is hingedly attached to one of the side panels.

37. The resposable container of claim 35 wherein the top panel is hingedly attached to one of the side panels and the top panel further comprises at least one flap extending from the top panel to cover at least one side panel and the exhaust vent therein.

38. The resposable container of claim 35 wherein the exhaust vent is defined by a series of perforations allowing the user to selectively open the exhaust vent.

39. The resposable container of claim 35 further comprising a rack elevating the contents of the container from the floor panel.

40. The resposable container of claim 39 wherein the rack has a number of passages allowing the sterilant to access the rack and the contents thereon.

41. A resposable container usable for storage, transport, disposal and sterilization of contents, the container comprising:
a) an enclosable area comprised of a series of side panels, a floor panel and a top panel;
b) at least one selectively openable exhaust vent in at least one of the panels; and
e) at least one of the panels substantially constructed of a material that is compatible with a sterilization process used to sterilize the contents of the container and where the sterilization process is chosen from a group comprising at least one of microwave, steam-microwave, electron beam irradiation, irradiation, ultraviolet light, dry heat, convection heat, convection steam, gamma irradiation, hydrogen peroxide, ethylene oxide, ozone, steam unwrapped method, steam gravity displacement wrapped method, steam pulse-vacuum wrapped or unwrapped, steam pre-vacuum wrapped or unwrapped, paracetic acid, chlorine dioxide, gas plasma, formaldehyde-low temperature steam, microwave—bactericide, xenon lamp, glass bead, vacuum ovens, heat conduction ovens, forced air ovens, solvent venting ovens, anprolene gas.

42. The resposable container of claim 41 wherein the top panel is hingedly attached to one of the side panels.

43. The resposable container of claim 41 wherein the top panel is hingedly attached to one of the side panels and the top panel further comprises at least one flap extending from the top panel to cover at least one side panel and the exhaust vent therein.

44. The resposable container of claim 41 wherein the exhaust vent is defined by a series of perforations allowing the user to selectively open the exhaust vent.

45. The resposable container of claim 41 further comprising a rack elevating the contents of the container from the floor panel. The resposable container of claim 45 wherein the rack has a number of passages allowing the sterilant to access the rack and the contents thereon.

46. The resposable container of claim 45 further comprising a tray providing support for the container and its contents.
